(19) United States
(12) Patent Application Publication
(10) Pub. No.: US 2004/0126449 A1
(43) Pub. Date: Jul. 1, 2004

(54) Topical composition and methods for treatment of aged or environmentally damaged skin

(76) Inventors: Gopa Majmudar, Irving, TX (US); Dawn Elizabeth Burke-Colvin, Dallas, TX (US); John Raymond Schiltz, Coppell, TX (US)

Correspondence Address: FULBRIGHT & JAWORSKI L.L.P., 600 CONGRESS AVE., SUITE 2400, AUSTIN, TX, 78701, US

(21) Appl. No.: 10/331,840

(22) Filed: Dec. 30, 2002

(51) Int. Cl. A61K 31/715; A61K 38/16

(52) U.S. Cl. 424/780; 514/8; 514/54

(57) ABSTRACT

The invention describes a unique composition of ingredients for improving the skin's visual appearance, function, and biophysical properties that has been changed by factors such as chronological age, chronic sun exposure, adverse environmental pollutants, household chemicals, disease pathologies, smoking, and malnutrition. In particular, the present invention relates to the composition of topically-applied cosmetics formulas that effectively treat aged and environmentally-damaged skin. These formulas may comprise various combinations of cosmetically compatible plant lectins, a high energy source for skin cell metabolism such as lactobacillus ferment, hydrolyzed wheat protein, glycine, arginine, and methionine and β-Glucans, which are formulated into different types of cosmetic vehicles. The invention provides for an effective alternative to the use of mono- and polyhydroxy acids, retinoid compounds, vitamins, nutraceuticals, physical corneum desquamating devices, and various plant and animal extracts.

### BACKGROUND OF THE INVENTION

1. Field of the Invention

The invention describes a unique composition of ingredients for improving the skin's visual appearance, function, and biophysical properties that have been changed by factors such as chronological age, chronic sun exposure, adverse environmental pollutants, household chemicals, disease pathologies, smoking, and malnutrition. In particular, the present invention relates to the composition of topically applied cosmetics formulas that effectively treat aged and environmentally damaged skin. These formulas contain various combinations of cosmetically compatible plant lectins, a high energy source for skin cell metabolism (such as lactobacillus ferment, hydrolyzed wheat protein, glycine, arginine, and methionine) and β-Glucans, which are formulated into different types of cosmetic vehicles. The invention provides for an effective alternative to the use of mono- and polyhydroxy acids, retinoid compounds, vitamins, nutraceuticals, physical corneum desquamating devices, and various plant and animal extracts and ferments.

2. Description of Related Art

With chronological age and chronic exposure to adverse environmental factors, the visual appearance and feel, physical properties, and physiological functions of skin change in ways that are considered cosmetically undesirable. The most notable and obvious changes include the development of fine line and wrinkles, loss of elasticity and firmness, increased sagging, loss of color evenness (tone), coarse surface texture, and mottled pigmentation. Less obvious, but measurable changes that occur as skin ages or endures chronic environmental insult include a general reduction in cellular and tissue vitality, reduction in cell replication rates, reduced cutaneous blood flow, reduced moisture content, accumulated errors in structure and function, and a reduction in the skin's ability to remodel and repair itself. Many of the above alterations in appearance and function are caused by changes in the outer epidermal layer of the skin, while others are caused by changes in the dermis.

When the epidermis is injured, the epidermal basal cells respond to the injury by dividing at a more frequent rate. This increase in replication rate results in a more rapid replacement of the damaged epidermis with a new epidermis and stratum corneum, a process referred to as “epidermal cell renewal.” Common examples of injuries that can increase epidermal cell renewal rates include abrasion, chemical damage, pH extremes, excessive sun exposure, or allergic or non-allergic contact irritation. If the injury is too severe, the increased replication will result in a “hyperplastic” epidermis and a thickened, poorly-functioning stratum corneum which is manifested as dry, rough scales. Other common stimuli which induce epidermal cell renewal include physical removal of the stratum corneum (i.e., an example of which is tape stripping, a process where tape is applied to the skin and pulled off, removing the top layer of the stratum corneum with it) and friction (i.e., on the soles and heels of the feet), all processes which result in epidermal hyperplasia.

With chronological age and chronic environmental exposure (notably UVA, UVB, and IR radiation), the dermis undergoes changes in structure and function which result in many of the characteristics of aged skin, including loss of elasticity, formation of wrinkles, loss of water-holding capacity, sagging, and poor microcirculation. At the molecular level, these changes have been correlated with biochemical changes in the content and structure of the extracellular matrix to which the major cells of the dermis (i.e., the fibroblasts) reside. Collagen becomes highly cross-linked and inelastic, elastin is reduced in amounts and is incorrectly distributed, and the glycosaminoglycans become reduced in amounts, which results in reduced intercellular water.

As a result of this changed architecture, the normal amounts and distribution of trace metal ions, growth factors, hormones, and cytokines becomes altered which causes the fibroblasts to become metabolically less active or quiescent. Although these cells have natural mechanisms to repair themselves and the matrix in which they reside, with age and too much damage, they are less able to repair the damage, and the condition continues to deteriorate. If the quiescent fibroblasts can be metabolically activated and stimulated to divide, they will synthesize new extracellular matrix and the old, damaged matrix will be enzymatically degraded and replaced. This process of balanced synthesis and degradation is referred to as “dermal remodeling.” The activation process can be accomplished in many different ways, including chemical stimulation by selected hormones, growth factors, cytokines, vitamins, botanical extracts and retinoids, or by increasing the nutrient supply (i.e., blood flow) to the tissue.

Although the mechanisms are not completely understood, it appears that physical or chemical changes to the intact stratum corneum of the skin will result in epidermal basal cell replication and subsequent increases in epidermal cell renewal. If the injury stimulus is too great, the skin will be unable to correct the damage or will “over-respond” in such a way as to cause extensive epidermal hyperplasia and dry, flaky, poorly-differentiated stratum corneum. If the damage stimulus is less and is well controlled, the process of epidermal replacement will result in a healthier, better-functioning epidermis and in a stratum corneum which looks and feels better, has greater capacity to hold moisture, and has fewer surface fine lines.

It is known that damage to the stratum corneum not only sets into motion natural biochemical mechanisms to repair and replace the epidermis, but disturbances in the corneum also stimulate repair and remodeling of the dermis. Prior art physiological, chemical, or mechanical methods of increasing stratum corneum renewal rates to achieve benefit such as hydroxy acids, retinoids, barrier disrupters, tape stripping, solvent extraction, etc. all have various drawbacks, such as significant irritation to the skin, skin toxicity, the requirement of high concentrations of expensive ingredients, or of low pH. In addition, all these methods can involve the invocation of damage to the skin, which may or may not set up adequate repair mechanisms to provide benefits to the skin.

For example, U.S. Pat. No. 5,720,963 to Smith (“the '963 patent”) discloses that chronic and significant disruption of the skin's water barrier using a combination of cerebrosides, hydroxy acids, and retinoids causes chronic injury to the corneum and results in epidermal and dermal repair of the structurally deteriorated skin if the disruption is maintained for a sufficient period of time. The '963 patent teaches that water barrier disruption agents such as cerebrosides or organic solvents or detergents, in combination with retinoids or hydroxy acids will disrupt the corneum water barrier and stimulate basal cell replication rates.

In a different approach, Schiltz discloses methods for treating aged and environmentally damaged skin in U.S. Pat. No. 6,495,126, which describes compositions comprising sufactants and chelating agents and uses of such compositions. The compositions and methods disclosed in U.S. Pat. No. 6,495,126 achieve repair and replacement of stratum corneum, epidermis, and dermis by means of corneum protease activation.

Damage to the skin due to exposure to bad weather, solar aggression, pollution, and mechanical impacts is counteracted by cell protective mechanisms of the skin. One such mechanism as identified by Greff (French Patent 2,725,896) involves phosphocreatine.

Phosphocreatine (also called creatine phosphate) is a phosphorylated molecule, generated from creatine and a phosphate group. The synthesis of phosphocreatine occurs by the enzyme creatine kinase, which transfers the phosphorus group of an ATP molecule to creatine. This enzyme is capable of metabolizing phosphocreatine into creatine and ATP. Phosphocreatine is a more stable molecule than ATP and it constitutes a reserve of energy that can be rapidly mobilized thus, ‘easy’ chemical energy intake is enabled. Phosphocreatine has been extensively studied and occurs in skin.

Specific compositions are known, in particular a certain type of fermentation liquor, enriched with the amino acids glycine, arginine and methionine (precursors necessary for synthesis of creatine) to be capable of stimulating the cells of the skin to synthesize phosphocreatine in situ. These compositions are therfore a high energy source for skin cell metabolism.

Applicant has found compositions and methods of treatment for aged and environmentally damaged skin that, surprisingly, enhances the stratum corneum turnover rate and improves skin condition. The treatment, which results in skin with improved visual appearance, function, and clinical/biophysical properties, is not known in the prior art. Moreover, the novel compositions and methods of treatment of the present invention accomplish this at low concentrations, at a neutral pH, in all vehicles and without causing clinical irritation or chronic damage to the skin.

### SUMMARY OF THE INVENTION

The features of the invention may be understood, made, and used by reference to the claims, the following description, and the description provided throughout the specification. Further features of the invention are apparent from the entirety of the specification or may be learned through the practice of the invention.

The invention provides a unique composition of ingredients for improving the skin's visual appearance, function, and biophysical properties that has been changed by factors such as chronological age, chronic sun exposure, adverse environmental pollutants, household chemicals, disease pathologies, smoking, and malnutrition. In particular, the present invention relates to the composition of topically-applied cosmetics formulas that effectively treat aged and environmentally-damaged skin. These formulas may contain various combinations of cosmetically compatible plant lectins, a high energy source for skin cell metabolism (such as lactobacillus ferment, hydrolyzed wheat protein, glycine, arginine, and methionine) and β-Glucans, which are included in different types of cosmetic vehicles such as would be well known to those of skill in the art. Such vehicles would include but not be limited to lotions, cremes, conditioners, sprays, roll-on solutions, and the like. The invention provides for an effective alternative to the use of mono- and polyhydroxy acids, retinoid compounds, vitamins, nutraceuticals, physical corneum desquamating devices, and various plant and animal extracts.

The present invention is directed to compositions comprising a chemically compatible combination comprising β-Glucans, plant lectins, and a high energy source for skin cell metabolism. β-Glucans appropriate for use in the invention, as known to those of skill in the art, may be sourced from commercial suppliers. In various embodiments, the plant lectins preferrably have a molecular weight of about 20,000 Daltons. Cosmetically compatible plant lectins are well known to those of skill in the art and may be commercially obtained. For example, DERMOLECTINE™ identifies the source of plant lectins that may be used in preferred embodiments. DERMOLECTINE™ is marketed by Sederma (Le Perray, France). In particular embodiments, the high energy source for skin cell metabolism comprises glycerin, Lactobacillus ferment, hydrolyzed wheat protein, glycine, arginine, and methionine. For example, PHOSPHOVITAL™ identifies the source of a high energy source for skin cell metabolism, marketed by Sederma (Le Perray, France) and is described in detail in French Patent No. 2,725,896.

β-glucans are marketed by various suppliers for use in cosmetics, recognizing their ability to strengthen the immunocompetence of the skin, for their protection against UV-induced skin aging, for their ability to support the regeneration of damaged cells, and for their ability to stimulate the formation of collagen and elastin fibers. β-Glucans can be used in the compositions of the invention in concentrations from 0.05% to 30% or more. By example, β-Glucan, from oats, is marketed by Dragoco Inc. (Totowa, N.J.) under the name of DRAGO-β-GLUCAN-OAT™.

In one aspect, the composition comprises plant lectins in final concentrations from 0.001% to 50%. Plant lectins are high molecular weight glycoproteins with molecular weights that range from 10,000 to 100,000 Daltons (Goldstein and Etzler, 1983). In a preferred embodiment, the plant lectins of the composition are of a molecular weight of about 20,000 Daltons. In a particular embodiment, the plant lectins are plant glycoproteins with a molecular weight of about 20,000 Daltons, purified from Solanum tuberosum L. (potato). For example, plant lectins marketed under the name of DERMOLECTINE™ by Sederma (Le Perray, France) are commercially available for use in making the present invention. This material can be used in formulas in concentrations from 0.05% to 30% or more.

In one aspect, the composition comprises a high energy source for skin cell metabolism. In an exemplary embodiment, the high energy source comprises Lactobacillus ferment. In a particular embodiment, the high energy source consists of phosphocreatine-derived molecules from biotech-modified microorganisms. It consists of glycerin, Lactobacillus ferment, hydrolyzed wheat protein, glycine, arginine, and methionine. By example, this energy source is commercially available under the name of PHOSPHOVITAL™ and is marketed by Sederma (Le Perray, France). This material provides a high energy source for skin cell metabolism. This material can be used in formulas in concentrations from 0.05% to 30% or more.

Compositions of the present invention include those in which the final concentration of the high energy source is about 3.00% by weight, the final concentration of plant lectins is 3.00% by weight, and the final concentration of β-Glucan is 2.00% by weight. An additional embodiment comprises a composition in which the final concentration of the high energy source is about 6.00% by weight, the final concentration of plant lectins is 8.00% by weight, and the final concentration of β-Glucans is 2.00% by weight. An additional embodiment comprises a composition in which the final concentration of the high energy source is about 6.00% by weight, the final concentration of plant lectins is 8.00% by weight, and the final concentration of β-Glucans is 2.00% by weight.

Embodiments of the present invention also include compositions comprising combinations in which the final concentration of the high energy source is from 0.05% to 30% by weight, the final concentration of plant lectins is 0.05% to 30% by weight, and the final concentration of β-Glucan is 0.05% to 30% by weight.

Further embodiments of the present invention comprise combinations in which the final concentration of high energy source is about 3.00% by weight, the final concentration of plant lectins is 3.00% by weight, and the final concentration of β-Glucan is 2.00% by weight. An additional embodiment comprises a combination in which the final concentration of high energy source is about 6.00% by weight, the final concentration of plant lectins is 8.00% by weight, and the final concentration of β-Glucans is 2.00% by weight.

The compositions and methods of the invention are effective to provide anti-aging benefits in all suitable cosmetic vehicles, including emulsions (oil in water, water in oil, water in oil in water, water in silicone), solutions (aqueous and hydro-alcoholic), suspensions, gels, ointments, and encapsulated particles in anhydrous systems. One skilled in the art would generally recognize these and other standard cosmetic vehicles that can be used in the present invention. It is important, however, that the concentrations and combinations of the ingredients be selected in such a way that the combinations are chemically compatible and do not form complexes which precipitate from the finished product.

Compositions prepared with appropriate and effective combinations of a high energy source, plant lectins, and β-Glucan can also contain other benefit agents, such as moisturizing agents (humectants and occlusive agents), anti-oxidants, sunscreens (UVA and UVB), skin lightening agents, hydroxy acids, emollients, anti-irritants, vitamins, trace metals, antimicrobial agents, botanical extracts, fragrances, and dyes and color ingredients.

In a most preferred embodiment, the final concentrations by weight of the ingredients of the composition are as shown in Table 1.

TABLE 1

| Ingredient | % In Formula |
|---|---|
| Water | 79.99 |
| Disodium EDTA | 0.10 |
| Ferulic Acid | 0.01 |
| Acrylates/C10-30 Alkyl | 0.38 |
| Acrylate Crosspolymer | |
| Butylene Glycol | 5.00 |
| Methylparaben | 0.20 |
| L-Arginine | 0.01 |
| Water | 5.00 |
| Triethanolamine, 99% | 0.35 |
| DMDM Hydantoin | 0.20 |
| Extracts of: Evening | 0.01 |
| Primrose, Jasmine, Anise | |
| and Buckbean | |
| Sea Rocket Extract | 0.01 |
| Sea Fennel Extract | 0.01 |
| High Eenergy Source | 3.00 |
| (e.g. PHOSPHOVITAL ™) | |
| Plant lectins (e.g. | 3.00 |
| DERMOLECTINE ™) | |
| β-Glucans | 2.00 |
| D&C Violet #2 | 0.08 |
| Lactose, Cellulose, Iron | 0.50 |
| Oxides, Titanium Dioxide, | |
| Retinyl Pamitate, Tocooheryl | |
| Acetate, Ascorbyl Palmitate | |

An additional embodiment of the present invention comprises methods of treating environmentally damaged or aged skin. These methods include regimens of treatment wherein the number of treatments in a 24 hour period is 1, 2, 3, 4, 5, 6, 7, 8, 9, 10, 11, 12, 13, 14, 15, 16, 17, 18, 19, 20, 30, 40, 50 to continuous treatment throughout the day and any range therein and at any convenient time interval between treatments. In a preferred embodiment, the treatment is combined with other treatments for the conditioning of skin or for the treatment of other conditions, either cosmetic or medical. A preferred embodiment comprises alternating treatment or alternating treatment regimes with the compositions of the present invention and a skin moisturizer.

In additional embodiments, the present invention contemplates cosmetic or dermopharmaceutical compositions which have a hair or scalp stimulating activity, characterized by the fact that they contain microbial filtrates enriched with glycine, arginine and methionine in essentially equal portions and that their in vivo cutaneous effect is stimulation of the synthesis of phosphocreatine. In further embodiments cosmetic or dermopharmaceutical compositions according to the present invention are characterized by the fact that the aforementioned microbial filtrates are obtained from fermentation liquors of bacteria, yeasts or molds. In still further embodiments, these compositions are characterized by the fact that the aforementioned microbial filtrates are obtained from fermentation liquors using strains chosen from the genera Lactobacillus, Saccharomyces or Aspergillus. In further embodiments, these compositions are characterized by the fact that the concentration of the three amino acids glycine, arginine and methionine is between 5 and 500 mM, and preferably between 20 and 100 mM. In still further embodiments, the cosmetic or dermopharmaceutical compositions are characterized by the fact that the microbial filtrates are used in liquid form or in dry form obtained by atomization, evaporation or lyophilization. In yet a further embodiment, these compositions are characterized by the fact that the concentration in terms of microbial filtrates is between 0.01 and 50% (wt/wt), and preferably between 0.5 and 10 wt % of the total composition.

In further embodiments of the invention, these compositions are characterized by the fact that the microbial filtrates are used in any form used in cosmetics or dermopharmacy. These compositions are further characterized by the fact that the microbial filtrates are incorporated in cosmetic carriers such as liposomes, chylomicrons, macro-, micro- and nanoparticles as well as macro-, micro- and nanocapsules, or absorbed using powdery organic polymers, talcs, bentonites and other mineral supports. In still further embodiments, the compositions are characterized by the fact that the microbial filtrates are combined in the cosmetic compositions with any other ingredient ordinarily used in cosmetics: extracted and/or synthetic lipids, gelling or viscosity-enhancing polymers, surfactants and emulsifiers, water-soluble or liposoluble active ingredients, plant extracts, tissue extracts, marine extracts In still yet a further embodiment, cosmetic or dermopharmaceutical compositions contemplated by the present invention, are characterized by the fact that the microbial filtrates are used in cosmetic applications for all treatments of the skin.

Following long-standing patent law, the words “a” and “an,” when used in conjunction with the word “comprising” or “comprises” in the claims or specification, denotes one or more.

### DETAILED DESCRIPTION OF THE INVENTION

The present invention provides a novel composition for treating aged and environmentally damaged or deteriorated skin. The present invention provides a method of treating skin comprising topical application to damaged skin of a cosmetic composition comprising consisting of a combination of cosmetically compatible plant lectins, a high energy source for skin cell metabolism such as lactobacillus ferment, hydrolyzed wheat protein, glycine, arginine, and methionine and β-Glucans, in a suitable vehicle, that is effective to induce repair, replacement, and remodeling of the stratum corneum, epidermis, and dermis of the skin and improvements in the appearance, function, and aging properties of the skin.

The effectiveness of the compositions and methods of the present invention in providing skin anti-aging benefits can be measured by a number of ways. Each of these strategies for evaluating the effectiveness of the invention can be used independently or together by one skilled in the art. The method is effective when it decreases stratum corneum turnover time by from about 1% to about 40%, preferably by at least about 12%. It is effective when it decreases skin dryness, surface fine lines, and canthus wrinkles. It is also effective when it increases firmness, smoothness, softness, clarity, neck texture, and face and neck moisture.

For example, the compositions and methods are effective when face moisture improves over a period of use. The period of use may range from several days, to eight weeks or more, or may be continuous. In one example, the compositions are effective to increase face moisture from about 24% to about 48% over a period of use of from about two to eight weeks. Similarly, the invention is effective to improve dryness over a period of use, which may range from several days to several weeks or may be continuous. In a particular example, the composition is effective to improve dryness from about 40% to about 63%, or when surface fine lines improve from about 24% to about 43% over a similar period of use. Likewise, the invention is effective when smoothness improves from about 30% to about 70% over the same period or when softness improves from about 32% to about 110% over a similar period. The invention is also effective when a majority of users report improvement in their skin, compared to when they began using the invention after as few as 2, and perhaps as many as 8 or more weeks of use. These effects are illustrative only and are not limiting as to the improvements or changes to be expected upon use of the present invention.

The cosmetic composition should be topically applied regularly to whatever skin area requires treatment with the frequency and in the amount necessary to achieve the desired results. In one example, the cosmetic composition is applied at least once per day and most preferably at night. The frequency of treatment depends on the degree of damage or deterioration of the skin, the responsiveness of the user's skin, the strength of the active ingredients in the cosmetic product, the effectiveness of the vehicle used to deliver the active ingredients into the stratum corneum, the ease with which the formula is removed by physical contact with clothing or it's removal by sweat or other intrinsic or extrinsic fluids, and the convenience to the user's lifestyle. Typical concentrations of biochemically active substances such as the novel treatment composition described herein can range from about 0.01% to about 10.0% by weight based on the total weight of the cosmetic composition, and the formula should be applied to the skin at a rate equal to from about 0.1 mg/cm² of skin to about 50.0 mg/cm² of skin.

The cosmetic composition of the present invention comprises safe and effective amounts of combinations of cosmetically compatible plant lectins, a high energy source for skin cell metabolism such as lactobacillus ferment, hydrolyzed wheat protein, glycine, arginine, and methionine and β-Glucans. The combinations may optionally include other ingredients to achieve the desired formulation of the combination.

The indicated combinations of compatible plant lectins, a high energy source for skin cell metabolism such as lactobacillus ferment, hydrolyzed wheat protein, glycine, arginine, and methionine and β-Glucans are effective in all suitable cosmetic vehicles, including emulsions, creams, lotions, solutions (both aqueous and hydro-alcoholic), anhydrous bases (such as lipsticks and powders), gels, and ointments. One skilled in the art would generally recognize these and other standard cosmetic vehicles that can be used in the present invention. Thus, the present invention may be formulated with a variety of cosmetic vehicles in addition to those described in the Examples below. Variations and other appropriate vehicles will be apparent to the skilled artisan and are appropriate for use in the present invention. Preferably, the cosmetic vehicle is selected from oil-in-water emulsions, hydro-alcoholic solutions, and encapsulated beads in anhydrous systems. Most preferably, the vehicle is an oil-in-water emulsion. Such emulsions and their compositions and methods of making are well known in the art. It is important, however, that the concentrations and combinations of the cosmetically compatible plant lectins, a high energy source for skin cell metabolism such as lactobacillus ferment, hydrolyzed wheat protein, glycine, arginine, and methionine and β-Glucans be selected in such a way that the combinations are chemically compatible and do not form complexes which precipitate from the finished product.

The composition of the present invention can be used in many cosmetic products including, but not limited to, moisturizing cream, skin benefit creams and lotions, gels, ointments, foundation, day or night cream, lipstick, cleansers, toners, masks, and color cosmetic products. The composition is most preferably used in anti-aging products for the face and other body parts, most especially leave-on products.

Compositions of the present invention that include suspension agents may include polyacrylic acid copolymers. For example, such copolymers are commercially available under the name Carbopol™ ETD 2020. Carbopol™ ETD 2020 is available from NoVeon, Incorporated, 9911 Brecksville Road, Cleveland, Ohio, USA. This material is a cross-linked polyacrylic acid copolymer in a toxicologically-preferred co-solvent system. Similar copolymers may be employed to achieve the purpose as will be appreciated by one of skill in the art.

Products according to the present invention in which antioxidant properties are desired may include ascorbic acid, propyl gallate, tocopheryl acetate, and other compounds and compositions with similar or functionally equivalent properties as is well known in the art. Products according to the present invention in which moisturizing properties are desired may include aloe barbadensis gel, glycereth-26, glycerin, sodium PCA, and other compounds and compositions with similar or functionally equivalent properties as is well known in the art.

Products according to the present invention in which ultraviolet light (UVA and UVB) absorbing properties are desired may include benzophenone-3, PABA, and other compounds and compositions with similar or functionally equivalent properties as is well known in the art.

As is well known in the skill of the art, additional skin care preparation ingredients include skin lightening agents (e.g. hydroquinine, preservatives (e.g. methylparaben, dmdm hydantoin), emollients (i.e. organic esters, di- and triglycerides, etc.), antiirritants (i.e., nonsteroidal antiinflammatories, glycyrrhizates, etc.), and fragrances (natural and artificial).

One skilled in the art will understand that the terms “mixture” and “mixing” in this patent are used in the broad sense of the words, with the term “mixing” including, but not limited to, stirring, blending, dispersing, milling, homogenizing, and other similar methods.

The cosmetic composition of the present invention is effective at pH values between pH 4 and pH 9. Preferably, the pH of the composition is between the following pH ranges: about 5.5 and about 6.5, about 4 to about 9, about 4 to about 8, about 4 to about 7, about 5 to about 9, about 5 to about 8, about 5 to about 7. Most preferably, the pH is about 6. One of ordinary skill in the art may add appropriate pH adjusting ingredients to the compositions of the present invention to adjust the pH to an acceptable range.

A high energy source as identified by Greff (French Patent, No. 2,725,896) is also contemplated for use in combination with other compositions of the present invention as a cosmetic treatment. In this study, it was discovered that a certain type of fermentation liquor, enriched with the amino acids glycine, arginine and methionine (precursors necessary for synthesis of creatine) is capable of stimulating the cells of the skin to synthesize phosphocreatine in situ. This was demonstrated using a strain of Lactobacillus casei grown on a conventional fermentation medium (containing mineral salts, yeast extract, peptone) and enriched with glycine, arginine and methionine in equal parts (50 mM) for 180 h. After cessation of growth and at the end of autolysis, the fermentation liquor is collected, filtered, decolorized, deodorized and optionally preserved. This product can be used in the present invention as a high energy source for skin cell metabolism.

Greff performed an in vivo study in order to demonstrate the effectiveness of this microbial filtrate. Phosphorus-3 1 nuclear magnetic resonance is a technique which, using surface probes, allows one to detect the concentration of phosphorylated molecules in the skin (ATP, phosphocreatine, inorganic phosphate). A placebo cream was tested versus a cream containing 5% of the microbial filtrate, over a duration of 6 h. The phosphorus-3 1 NMR study showed that the concentration of inorganic phosphate (metabolic “waste”) increased in the course of the day at the sites receiving the placebo, but that it decreased on the sites treated with the cream containing the microbial filtrate. Concomitantly, the concentration of phosphocreatine decreased at the placebo sites and increased at the treated sites.

Greff discloses that the type of microbial filtrate is not limited to the lactobacilli or to the bacteria; it can also be obtained from the fermentation liquors of yeasts or molds, for example. A skilled artisan in the field of the fermentation processes will be able to find and to choose the microorganism most suitable for the job. The genera of Lactobacillus, Saccharomyces or Aspergillus are particularly recommended. The main objective is to obtain a product which stimulates the synthesis of phosphocreatine in the skin. The quantities of the amino acids such as glycine, arginine and methionine which are added to the base fermentation medium can vary between 5 and 500 mM/L, and preferably between 20 and 100 mM/L. The respective proportions of these three amino acids can vary between 0.5 and 2 parts for each.

Greff discloses that the microbial filtrates thus obtained can be used in cosmetic and dermopharmaceutical products of any sort, preferably in the products intended for the treatment of tired, exposed skin, treatment of the hair and of the scalp as well as for stimulation of hair growth.

The main use of these microbial filtrates is therefore as a high energy source for skin cell metabolism. For example, for stimulation of the growth of cells, and for their metabolism and synthesis of phosphocreatine (reserve of energy); for application to the skin (face, hands, body). In other instances, the filtrates described may contribute to restructuring of the epidermis, improving the appearance of the skin, and for giving elasticity and firmness to tired skin. These compositions may therefore be used in products intended for different cosmetic and dermopharmaceutical treatments of the skin including refirming, toning, antiwrinkle treatment, for hydration and for the smoothing effect, and for the treatment of greasy skin and skin with acne. These microbial filtrates may also be very useful in hair products such as for hair care, prevention of hair loss, antidandruff effect, and improvement of the appearance and health of the hair (shininess, strength, flexibility).

Greff discloses that the microbial filtrates which are described, can be used in liquid form or in dry form obtained by atomization, evaporation or lyophilization. The microbial filtrates can be used in any galenic form used in cosmetics or dermopharmacy such as in oil-in-water emulsions and water-in-oil emulsions, milks, lotions, gels, ointments, body oils, hair lotions, shampoos, soaps, sticks and pencils, and sprays, but are not limited to such It is possible to incorporate the described microbial filtrates in cosmetic carriers such as liposomes, chylomicrons, macro-, micro- and nanoparticles as well as macro-, micro- and nanocapsules, and to absorb them using powdery organic polymers, talcs, bentonites and other mineral supports.

The concentration of these microbial filtrates can vary between 0.01 and 50% (wt/wt), and preferably between 0.5 and 10 wt % of the total composition of the finished product. Below these values, the effects are negligible, and above them, it is difficult to formulate an acceptable cosmetic product.

The microbial filtrates can be combined in cosmetic compositions with any other ingredient ordinarily used in cosmetics such as: extracted and/or synthetic lipids, gelling or viscosity-enhancing polymers, surfactants and emulsifiers, water-soluble or liposoluble active ingredients, extracts of other plants, tissue extracts, and marine extracts but are not limited to such.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Preparation of Compositions

Compositions comprising a chemically compatible combination comprising β-Glucans, cosmetically compatible plant lectins (e.g. DERMOLECTINE™) and a high energy source for skin cell metabolism (e.g. PHOSPHOVITAL™) may be made through the following steps. Ingredients are listed individually, followed by their percent by weight achieved in the final composition.

Step 1: Mix Butylene Glycol (5.00%) and Methylparaben (0.20%).

Step 2: Provide water (79.99%).

Step 3: Add the products of Step 1 with the water of Step 2.

Step 4: Add Disodium EDTA (0.10%) and mix.

Step 5: Add Ferulic Acid (0.01%) and mix.

Step 6: Add Carbopol™ ETD 2020 (0.38%) and mix.

Step 7: Add L-Arginine (0.01%) to water (5.00%) and mix.

Step 8: Add the products of Step 7 to the products of Step 6.

Step 9: Add 99%Triethanolamine (0.35%) to the products of Step 8 and mix.

Step 10: Add DMDM Hydantoin (0.20%) to the products of Step 9 and mix.

Step 11: Add Vegetch Night Breeze™ (0.01%) to the products of Step 10 and mix.

Step 12: Add Sea Rocket Extract (0.01%) to the products of Step 11 and mix.

Step 13: Add Sea Fennel Extract (0.01%) to the products of Step 12 and mix.

Step 14: Add high energy source e.g. PHOSPHOVITAL™ (3.00%) to the products of Step 13 and mix.

Step 15: Add plant lectin e.g. DERMOLECTINE™ (3.00%) to the products of Step 14 and mix.

Step 16: Add β-Glucan (2.00%) to the products of Step 15 and mix.

Step 17: Add D&C Violet #2 (0.08%) to the products of Step 16 and mix.

Step 18: While continuously mixing the products of Step 17, add Unispheres™ PACE (0.50%) at a rate sufficient to prevent precipitation or the settlement of solids.

Upon completion of the above steps, a composition of the ingredients is formed as listed in Table 2.

TABLE 2
Exemplary composition comprising β-Glucan, plant
lectin and high energy source in a suitable vehicle.

| Phase | Ingredient | % In Formula |
|---|---|---|
| A | Water | 79.99 |
| A | Disodium EDTA | 0.10 |
| A | Ferulic Acid | 0.01 |
| A | Acrylates/C10-30 Alkyl | 0.38 |
| | Acrylate Crosspolymer | |
| B | Butylene Glycol | 5.00 |
| B | Methylparaben | 0.20 |
| C | L-Arginine | 0.01 |
| C | Water | 5.00 |
| D | Triethanolamine, 99% | 0.35 |
| E | DMDM Hydantoin | 0.20 |
| F | Extracts of: Evening | 0.01 |
| | Primrose, Jasmine, | |
| | Anise and Buckbean | |
| F | Sea Rocket Extract | 0.01 |
| F | Sea Fennel Extract | 0.01 |
| F | High Energy Source | 3.00 |
| | (e.g. PHOSPHOVITAL ™) | |
| F | Plant Lectin (e.g. | 3.00 |
| | DERMOLECTINE ™) | |
| F | β-Glucan | 2.00 |
| F | D&C Violet #2 | 0.08 |
| G | Lactose, Cellulose, | 0.50 |
| | Iron Oxides, Titanium | |
| | Dioxide, Retinyl Pamitate, | |
| | Tocooheryl Acetate, | |
| | Ascorbyl Palmitate | |

### Example 2

Stratum Corneum Turnover Studies

Many anti-aging formulas and ingredients have the ability to activate the epidermis and increase the rate at which this tissue is replaced. The following procedure was utilized to estimate increased stratum corneum turnover rates on human skin, which can result directly from epidermal activation. As many as 5 different sites per forearm were marked using a plastic template, and baseline readings of color intensity were determined using a Minolta Chromameter (b* value). Occlusive Hilltop chambers (2 cm diameter) containing 0.05 ml Mary Kay Sun Essentials™ Sunless Tanning Lotion product with dihydroxyacetone (DHA) were placed on the sites. After 6 hours, these patches were removed, and 18 hours later, the color intensity was again determined using the Chromameter. The delta b* values were calculated as the difference between the reading and the baseline. Panelists themselves applied the compositions of the present invention to the brown spots in the morning and evening during the ensuing 10 days. The composition contained 3% high energy source (e.g. PHOSPHOVITAL™)+3% plant lectin (e.g. DERMOLECTINE™)+2% β-Glucan in the same vehicle as in Example 1. Chromameter readings were repeated after 4, 7, and 10 days. The color decay slope was calculated as the percent loss per day, and the transit time determined by extrapolating to 100% loss of color.

The results of this study (Table 3) clearly show that the combination of the high energy source+plant lectin, and β-Glucan increased the rate at which the stratum corneum replaced itself, compared to the vehicle that was used to incorporate these three materials. Furthermore, the effects were concentration-dependent. These increases in stratum corneum replacement rate provide direct evidence that the Applicants' Invention activates or stimulates the epidermis of the skin.

TABLE 3
Effects of a high energy source, plant lectin, and
β-Glucan on Human Stratum Corneum Turnover Rate.

| | | % Change in |
|---|---|---|
| | | Stratum Corneum |
| | Stratum Corneum | Renewal Rate vs. |
| | Renewal Rate | No Treatment |
| Product Tested | (Δ b*/Day) | Control |
| Untreated | 0.590 | — |
| Vehicle | 0.632 | 7.1 |
| Vehicle + 3% high | 0.666 | 12.9 |
| energy source (e.g. | | |
| PHOSPHOVITAL ™) + 3% | | |
| plant lectin (e.g. | | |
| DERMOLECTINE ™) + 2% | | |
| β-Glucan | | |
| Vehicle + 6% high | 0.691 | 17.1 |
| energy source (e.g | | |
| PHOSPHOVITAL ™) + 8% | | |
| plant lectin (e.g | | |
| DERMOLECTINE ™) + 2% | | |
| β-Glucan | | |

### Example 3

Long Term Anti-Aging Effects of The Invention On Human Facial Skin

The demonstration by the Applicants that a combination of high energy source (e.g. PHOSPHOVITAL™), plant lectin (e.g. DERMOLECTINE™), and β-Glucan increased the replacement rate of the stratum corneum is evidence that the materials activated the epidermis. It is well known that materials or finished formulas that activate the epidermis often provide long-term anti-aging benefits to the skin. Although the mechanisms involved are unknown, it is known that the epidermis can produce cytokines and growth factors that can stimulate the dermis to remodel itself. The following study was conducted to determine if the combination of the present invention provides long-term visual and measurable anti-aging benefits on the human face. For the study, 20 panelists applied the product to their skin as a part of their regular evening skincare routine. The composition contained 3% high energy source (e.g. PHOSPHOVITAL™)+3% plant lectin (e.g. DERMOLECTINE™)+2% β-Glucan in the same vehicle as in Example 1. During the day they applied a commercial moisturizer (Mary Kay Day Solution™ with SPF 15, marketed by Mary Kay, Inc.).

The panelists were monitored for skin condition at the beginning of the study (i.e. before treatment), and after 2, 4, and 8 weeks. They were evaluated for face and neck moisture, dryness, surface fine lines, canthus wrinkles, firmness, smoothness, softness, clarity, and neck texture. Face and neck moisture were evaluated using impedance measurements, an electrical conductivity measurement using the Nova Dermal Phase Meter. Dryness, surface fine lines, smoothness, and softness were determined by an expert grader using a calibrated visual analog scale from 1 to 10. Skin smoothness and softness were evaluated using tactile observations by the grader. Surface fine lines were counted and the severity of the lines evaluated according to the Packman-Gans method, J. Soc. Cosmetic Chem. 29:70 (1978), using weighted scoring. Dryness was evaluated using a calibrated visual analog scale from 1 to 10.

Firmness was evaluated using a Hargens ballistometer, a device that evaluates the elasticity and firmness of the skin by dropping a small body onto the skin and recording its first two rebound peaks. As firmness decreases, the second peak will be smaller in comparison to the first. Clarity was evaluated using a Minolta Chromameter, which measures the total light reflected from the skin compared to the amount of red and brown/yellow light. These measurements were mathematically analyzed to determine the clarity of the skin.

Canthus wrinkles and neck texture were evaluated after 2, 4, and 8 weeks of product use by comparing the silicone replicas (negative impressions) made of the individuals' skin at baseline. The replicas were evaluated by computer image analysis to determine the number and depth of the wrinkles.

As shown in Table 4, continued improvement was seen for the skin condition parameters throughout the 8 weeks of the study. The composition used for the study is listed in Example 1. The composition was applied only at night by the panelists, and during the day they applied a commercial moisturizer (Mary Kay Day Solution™ with SPF 15).

TABLE 4
Effects of high energy source, plant lectin,
and β-Glucan on Human Skin Condition.

| | % Improvement Compared to Baseline | | |
|---|---|---|---|
| Skin Benefit | Week 2 | Week 4 | Week 8 |
| Face Moisture | 24.6 | 34.0 | 48.4 |
| Neck Moisture | 19.1 | 29.1 | 43.4 |
| Dryness | 40.1 | 52.1 | 63.1 |
| Surface Fine Lines | 24.4 | 33.2 | 43.2 |
| Canthus Wrinkles | 18.0 | 31.8 | 51.1 |
| Cheek Firmness | 14.2 | 23.6 | 33.5 |
| Neck Firmness | 11.3 | 18.6 | 27.7 |
| Smoothness | 30.6 | 47.1 | 69.4 |
| Softness | 32.0 | 68.8 | 110.4 |
| Clarity | 6.1 | 9.2 | 15.2 |
| Neck Texture | 7.6 | 12.3 | 25.1 |

### Example 4

Panelist Self Assessment of their Skin Condition When Using the Invention.

A composition of the invention was applied only at night by the panelists, and during the day they applied a commercial moisturizer (Mary Kay Day Solution™ with SPF 15). The composition used for the study is listed in Example 1. The values in Table 5 reflect the percentage of panelists who assessed improvement in their skin, compared to when they began using the product, based on a 5-point scale where 3 represented no change, 4 and 5 worsening, and 1 and 2 represented improvement.

TABLE 5
Panelist Self Assessment of their Skin
Condition When Using the Invention.

| Skin Condition Assessed | 2 Weeks | 4 Weeks | 8 Weeks |
|---|---|---|---|
| Dryness | 100 | 100 | 100 |
| Smoothness | 95 | 100 | 100 |
| Fine Lines and | 80 | 90 | 100 |
| Wrinkle Appearance | | | |
| Firmness | 90 | 95 | 100 |
| Softness | 100 | 100 | 100 |
| Healthy Glow | 70 | 75 | 100 |
| Looks and Feels Younger | 90 | 95 | 100 |
| Elasticity | 80 | 95 | 100 |
| Overall Skin Improvement | 90 | 100 | 100 |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A cosmetic treatment composition for aged or damaged skin comprising a combination of β-Glucans, plant lectins, and a high energy source for skin cell metabolism.

2. The composition of claim 1, comprising a concentration by weight of β-Glucans of 0.05% to 30%, a concentration by weight of plant lectins of 0.05% to 30%, and a concentration by weight of the high energy source for skin cell metabolism of 0.5% to 10%.

3. The composition of claim 2, wherein the concentration by weight of plant lectins is at least 3%.

4. The composition of claim 3, wherein the concentration by weight of plant lectins is at least 8%.

5. The composition of claim 2, wherein the high energy source for skin cell metabolism comprises glycerin, Lactobacillus ferment, hydrolyzed wheat protein, glycine, arginine, and methionine.

6. The composition of claim 5, wherein the concentration is at least 3%.

7. The composition of claim 5, wherein the concentration is at least 6%.

8. The composition of claim 2, wherein the concentration of β-Glucans is at least 2%.

9. The composition of claim 2, wherein the concentration by weight of β-Glucans is at least 2%, the concentration by weight of plant lectins is at least 3%, and the concentration by weight of the high energy source for skin cell metabolism is at least 3%.

10. The composition of claim 2, wherein the concentration by weight of β-Glucans is 2%, the concentration by weight of plant lectins is 8%, and the concentration by weight of the high energy source for skin cell metabolism is 6%.

11. A cosmetic treatment composition for aged or damaged skin comprising β-Glucans in a concentration by weight of 2%, plant lectins in a concentration by weight of 3%, and a high energy source for skin cell metabolism in a concentration by weight of 3%.

12. A method of treating skin comprising topical application to aged or damaged skin of a composition comprising a combination of β-Glucans, plant lectins, and a high energy source for skin cell metabolism.

13. The method of claim 12, wherein the composition is further defined as comprising a concentration by weight of β-Glucans of 0.05% to 30%, a concentration by weight of plant lectins of 0.05% to 30%, and a concentration by weight of the high energy source for skin cell metabolism of 0.5%to 10%.

14. The method of claim 13, wherein the concentration by weight of β-Glucans is at least 2%, the concentration by weight of plant lectins is at least 3%, and the concentration by weight of the high energy source for skin cell metabolism is at least 3%.

15. The method of claim 12, wherein the composition is applied at least once per day.

16. The method of claim 12, wherein the composition is applied at least once per day and a moisturizer is applied at least once per day.

17. The method of claim 16, wherein the composition is applied at least once per day before sleep and the moisturizer is applied at least once per day at waking.
